(19) United States
(12) Patent Application Publication
(10) Pub. No.: US 2014/0088411 A1
(43) Pub. Date: Mar. 27, 2014

(54) CATHETER INSERTION SUPPORTING SYSTEM, CATHETER INSERTION SUPPORTING METHOD, COMPUTER READABLE STORAGE MEDIUM STORED WITH PROGRAM FOR CATHETER INSERTION SUPPORTING SYSTEM, AND CALIBRATION METHOD

(71) Applicant: TERUMO KABUSHIKI KAISHA, Shibuya-ku (JP)

(72) Inventors: Satoru SUEHARA, Ashigarakami-gun (JP); Ryosuke Yamazaki, Fujinomiya-shi (JP); Makoto Narita, Ashigarakami-gun (JP)

(73) Assignee: TERUMO KABUSHIKI KAISHA, Shibuya-ku (JP)

(21) Appl. No.: 14/036,702

(22) Filed: Sep. 25, 2013

Related U.S. Application Data
(60) Provisional application No. 61/705,855, filed on Sep. 26, 2012.

(51) Int. Cl. A61B 5/00; A61B 5/06

(52) U.S. Cl. CPC A61B 5/0086; A61B 5/065
USPC 600/424; 600/425

(57) ABSTRACT

There is provided a catheter insertion supporting system including an extraction section configured to extract a contour shape of at least one of organs of a subject represented on a two-dimensional image picked up by an image pickup element of a catheter inserted in the body of the subject; a detection section configured to detect, in a three-dimensional CT image of the subject, a cross section which includes a contour shape which at least partly coincides with the contour shape extracted by the extraction section; and a display section configured to display a portion of the three-dimensional CT image which includes an organ having the cross section detected by the detection section together with the two-dimensional image.

### BACKGROUND

1. Technical Field

The present invention relates to a catheter insertion supporting system, a catheter insertion supporting method, a computer readable storage medium stored with a program for the catheter insertion supporting system, and a calibration method.

2. Description of Related Art

Inflammation caused in the nasal cavity by the cold, allergy or the like sometimes spreads to a paranasal sinus which is a cavity in a bone neighboring with the nasal cavity. The inflammation caused in the paranasal sinus in this manner is called sinusitis. The paranasal sinus is communicated with the nasal cavity through a small hole called natural ostium. However, if swelling or thickening of a mucous membrane occurs in the proximity of the natural ostium by the sinusitis, then the natural ostium is constricted. Therefore, secretion, bacteria and so forth in the paranasal sinus becomes less likely to discharge to the nasal cavity, and ventilation failure occurs.

Conventionally, as a treatment for the sinusitis, a surgical procedure of removing a constriction portion which constricts the natural ostium by forceps and a drill or the like is used popularly. However, in recent years, attention is paid to a minimally invasive procedure of expanding the constricted natural ostium by a balloon catheter. For example, Unexamined Japanese Patent Publication No. 2008-513125 discloses that a natural ostium is expanded by endoscopically manipulating a balloon catheter (hereinafter referred to merely as “catheter”).

However, if the catheter is manipulated based on an image of a camera provided at the distal end of the catheter, then this provides a problem that an organ in the nasal cavity makes an obstacle and makes it difficult to grasp the position of the natural ostium.

### SUMMARY

The present invention solves such problems as described above. In particular, according to the present invention, a contour shape of an organ is extracted from a two-dimensional image of a catheter from a camera, and a cross section including a contour shape which coincides with the extracted contour shape is detected in a three-dimensional CT image. Then, the three-dimensional CT image at the portion including an organ having the cross section detected is displayed together with the two-dimensional image from the camera. Consequently, it is possible to stereoscopically grasp the organ and its peripheral region displayed in the two-dimensional image from the three-dimensional CT image and easily grasp the position of a natural ostium which is difficult to confirm from the two-dimensional image of the catheter from the camera.

In the meantime, in the present invention, after a three-dimensional CT image of a subject is stored, the position and the direction of a catheter in the body are detected. Then, a three-dimensional image of an external form of the catheter is synthesized with the three-dimensional CT image by combining the three-dimensional image into the three-dimensional CT image such that the position and the direction, in the three-dimensional CT image, of the external form of the catheter of the three-dimensional image coincide with the detected position and direction of the catheter in the body, respectively. Then, the synthesized image is displayed together with the two-dimensional image of the catheter from the camera. Consequently, it is possible to stereoscopically grasp the positional relationship between the position of the distal end of the catheter and the natural ostium and stereoscopically grasp the organ and its peripheral region displayed in the two-dimensional image through the three-dimensional CT image.

In order to achieve at least one of the objects described above, the present invention includes the followings.

(1) A catheter insertion supporting system including an extraction section configured to extract a contour shape of at least one of organs of a subject represented on a two-dimensional image picked up by an image pickup element of a catheter inserted in the body of the subject, a detection section configured to detect, in a three-dimensional CT image of the subject, a cross section which includes a contour shape which at least partly coincides with the contour shape extracted by the extraction section, and a display section configured to display a portion of the three-dimensional CT image which includes an organ having the cross section detected by the detection section together with the two-dimensional image.

(2) A catheter insertion supporting method, including an extraction step of extracting a contour shape of at least one of organs of a subject represented on a two-dimensional image picked up by an image pickup element of a catheter inserted in the body of the subject, a detection step of detecting, in a three-dimensional CT image of the subject, a cross section which includes a contour shape which at least partly coincides with the contour shape extracted at the extraction step, and a display step of displaying a portion of the three-dimensional CT image which includes an organ having the cross section detected at the detection step together with the two-dimensional image of the organs.

(3) A non-transitory computer-readable storage medium stored with a program for a catheter insertion supporting system, the program causing the catheter insertion supporting system to execute a process including an extraction step of extracting a contour shape of at least one of organs of a subject represented on a two-dimensional image picked up by an image pickup element of a catheter inserted in the body of the subject, a detection step of detecting, in a three-dimensional CT image of the subject, a cross section which includes a contour shape which at least partly coincides with the contour shape extracted at the extraction step, and a display step of displaying a portion of the three-dimensional CT image which includes an organ having the cross section detected at the detection step together with the two-dimensional image of the organs.

(4) A catheter insertion supporting system including a storage section configured to store a three-dimensional CT image of a subject, a detection section configured to detect a position and a direction of a catheter inserted in the body of the subject in the body, a synthesis section configured to synthesize a three-dimensional image of an external form of the catheter with the three-dimensional CT image stored in the storage section by combining the three-dimensional image into the three-dimensional CT image such that a position and a direction of the external form of the catheter of the three-dimensional image in the three-dimensional CT image coincide with a position and a direction in the body of the catheter detected by the detection section, respectively, and a display section configured to display the three-dimensional CT image synthesized by the synthesis section together with a two-dimensional image picked up by an image pickup element of the catheter.

(5) A catheter insertion supporting method, including a storage step of storing a three-dimensional CT image of a subject, a detection step of detecting a position and a direction of a catheter inserted in the body of the subject in the body, a synthesis step of synthesizing a three-dimensional image of an external form of the catheter with the three-dimensional CT image stored at the storage step by combining the three-dimensional image into the three-dimensional CT image such that a position and a direction of the external form of the catheter of the three-dimensional image in the three-dimensional CT image coincide with a position and a direction in the body of the catheter detected at the detection step, respectively, and a display step of displaying the three-dimensional CT image synthesized at the synthesis step together with a two-dimensional image picked up by an image pickup element of the catheter.

(6) A non-transitory computer-readable storage medium stored with a program for a catheter insertion supporting system, the program causing the catheter insertion supporting system to execute a process including a storage step of storing a three-dimensional CT image of a subject, a detection step of detecting a position and a direction of a catheter inserted in the body of the subject in the body, a synthesis step of synthesizing a three-dimensional image of an external form of the catheter with the three-dimensional CT image stored at the storage step by combining the three-dimensional image into the three-dimensional CT image such that a position and a direction of the external form of the catheter of the three-dimensional image in the three-dimensional CT image coincide with a position and a direction in the body of the catheter detected at the detection step, respectively, and a display step of displaying the three-dimensional CT image synthesized at the synthesis step together with a two-dimensional image picked up by an image pickup element of the catheter.

(7) A calibration method, including an extraction step of extracting a contour shape of at least one of organs of a subject represented in a two-dimensional image picked up by an image pickup element of a catheter inserted in the body of the subject, a detection step of detecting, in a three-dimensional CT image of the subject, a cross section having a contour shape which at least partly coincides with the contour shape extracted at the extraction step, a calculation step of calculating a relative positional relationship between the organ having the cross section detected in the three-dimensional CT image at the detection step and the image pickup element of the catheter based on the two-dimensional image and specifications of a field of view of the image pickup element, and a calibration step of specifying a position of the image pickup element of the catheter on a coordinate system of the three-dimensional CT image based on the positional relationship calculated at the calculation step and the position of the organ in the three-dimensional CT image thereby to adapt the coordinate system of the three-dimensional CT image and the position of the image pickup element of the catheter to each other.

The above and other objects, features and advantages of the present invention will become apparent by referring to the preferred embodiments exemplified the following description and the accompanying drawings.

### BRIEF DESCRIPTION OF THE DRAWINGS

FIG. 1 is a block diagram depicting a configuration of a catheter insertion supporting system according to a first embodiment of the present invention;

FIG. 2 is a side elevational view of a catheter;

FIG. 3 is a flow chart illustrating a catheter insertion supporting method according to the first embodiment of the present invention;

FIG. 4 is a view depicting, in a simplified form, an anatomical structure of the inside of the nose of a patient having a constriction portion formed in a natural ostium communicating with a maxillary sinus which is one of the paranasal sinuses;

FIG. 5 is a block diagram depicting a configuration of a catheter insertion supporting system according to a second embodiment of the present invention;

FIG. 6 is a flow chart illustrating a catheter insertion supporting method according to the second embodiment of the present invention;

FIG. 7 is a block diagram depicting a configuration of a catheter insertion supporting system according to a third embodiment of the present invention;

FIG. 8 is a flow chart illustrating a catheter insertion supporting method according to the third embodiment of the present invention; and

FIGS. 9A and 9B are explanatory views illustrating different states in which a three-dimensional CT image synthesized with a three-dimensional image of an external form of a catheter is displayed together with a two-dimensional image of the catheter from an image pickup element.

### DETAILED DESCRIPTION

### First Embodiment

In the following, a catheter insertion supporting system, a catheter insertion supporting method, a computer readable storage medium stored with a program for the catheter insertion supporting system, and a calibration method according to a first embodiment of the present invention are described in detail below with reference to the drawings.

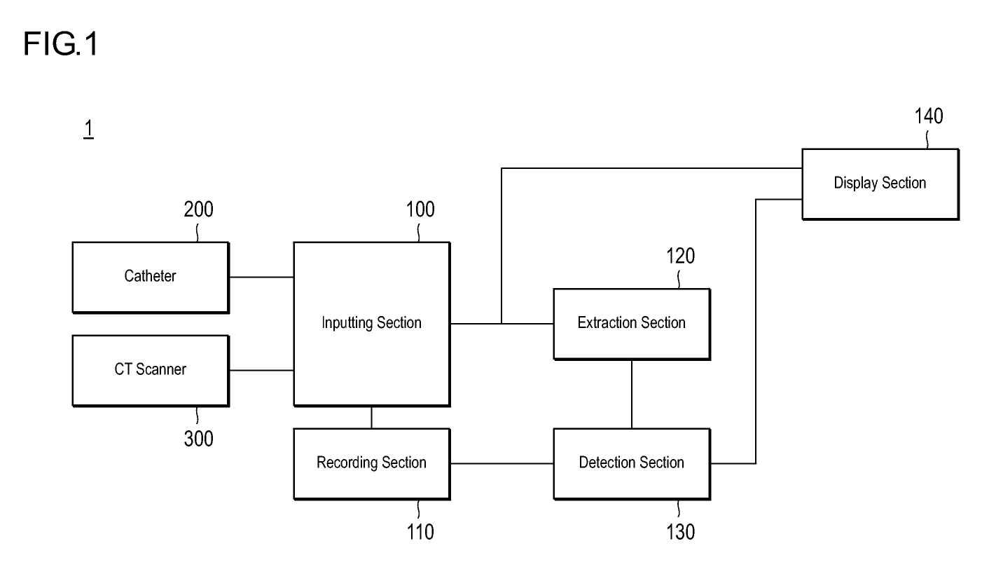

FIG. 1 is a block diagram depicting a configuration of the catheter insertion supporting system according to the first embodiment of the present invention.

The catheter insertion supporting system 1 according to the present embodiment includes an inputting section 100, a recording section 110, an extraction section 120, a detection section 130, a display section 140, a catheter 200, and a CT (Computed Tomography) scanner 300. The inputting section 100, the recording section 110, the extraction section 120, the detection section 130 and the display section 140 can be configured of a computer and a program.

The catheter 200 is a tube for medical use which is inserted into a body cavity such as a nasal cavity or a thoracic cavity, a lumen such as a digestive tract or a urinary duct, a blood vessel or the like. The catheter 200 has an image pickup element provided at the distal end thereof and capable of picking up a two-dimensional image. The catheter 200 is inserted, for example, into a nasal cavity of a patient and used for treatment of the sinusitis.

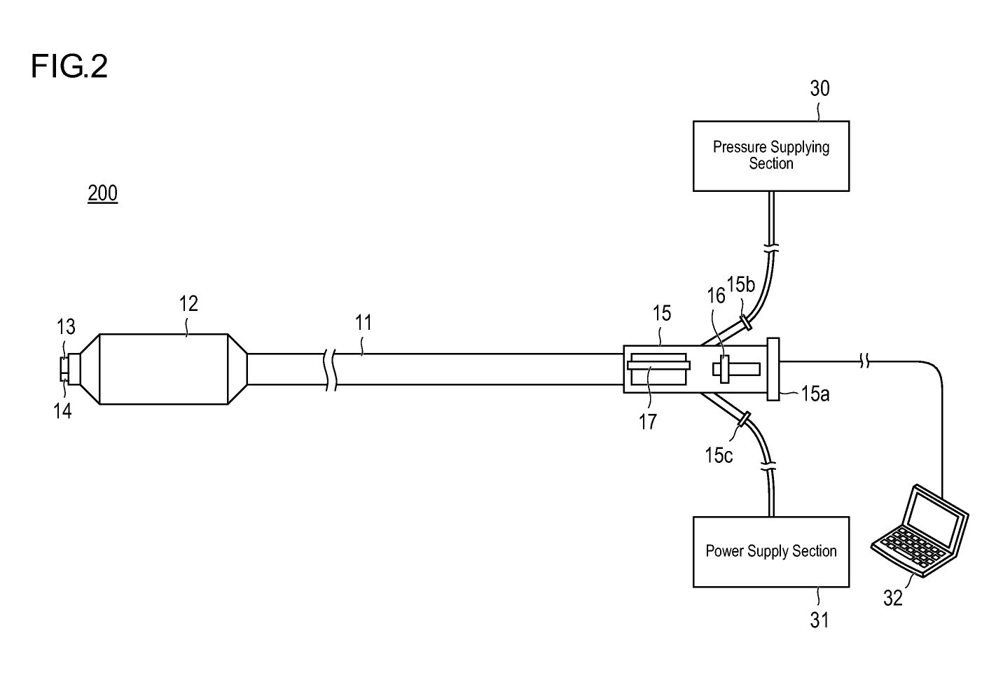

FIG. 2 is a side elevational view of a catheter. It is to be noted that FIG. 2 depicts, in addition to the catheter 200, a pressure supplying section 30, a power supply section 31 and a PC (Personal Computer) 32 having the display section 140, which are connected to the catheter 200.

The catheter 200 includes an elongated member 11, a balloon 12, a camera 13, a light irradiation section 14, and a hub 15 having a camera position operation section 16 and a curve operation portion 17. The elongated member 11 is a main body of the catheter 200. The balloon 12 is an expansion member for expanding a constriction portion of a natural ostium. The camera 13 is an image pickup element for picking up a two dimensional image in the nasal cavity. The light irradiation section 14 irradiates light in the nasal cavity. The camera position operation section 16 is configured to manipulate the position of the camera 13. The curve operation portion 17 is used to carry out a curving operation of a curved portion which is curved at a distal end portion of the elongated member 11. The hub 15 has an image signal connection portion 15a, a pressure supply connection portion 15b, and a power supply connection portion 15c. The image signal connection portion 15a is connected to the PC 32 so as to transmit a two-dimensional image picked up by the camera 13 to the PC 32. The pressure supply connection portion 15b is connected to the pressure supplying section 30 so as to supply liquid or the like to be supplied to the balloon 12. The power supply connection portion 15c is connected to the power supply section 31 so as to supply electric power to be supplied to the light irradiation section 14.

The camera 13 is provided at the distal end of the elongated member 11 movably forward and backward with respect to the direction of the longitudinal axis of the elongated member 11. The position of the camera 13 is moved forwardly and backwardly with respect to the direction of the longitudinal axis of the elongated member 11 when the camera position operation section 16 is manipulated by a doctor or the like. The camera 13 may be configured, for example, of a CCD (Charge Coupled Device) camera or a CMOS (Complementary Metal-Oxide Semiconductor) image pickup device. Alternatively, the camera 13 may be configured of an image fiber device formed by a plurality of bundled optical fibers for optically transmitting the two-dimensional image or a relay lens group formed from optical lenses and so forth for propagating a two-dimensional image.

It is to be noted that the camera 13 may not necessarily be provided movably forward and backward with respect to the direction of the longitudinal axis of the elongated member 11, but may otherwise be merely fixed to the distal end portion of the elongated member 11.

The two-dimensional image picked up by the camera 13 can be transmitted to the PC 32 connected to the image signal connection portion 15a and displayed on the display section 140 of the PC 32.

The light irradiation section 14 is provided at the distal end of the elongated member 11 and emits light when electric power is supplied thereto from the power supply section 31 connected to the power supply connection portion 15c. The light emitted from the light irradiation section 14 is irradiated forwardly of the light irradiation section 14. The light irradiation section 14 may be configured, for example, of an LED (Light Emitting Diode), a halogen lamp or an HID (High Intensity Discharge) lamp.

It is to be noted that the LED or a like element may not be provided at the distal end of the elongated member 11. In particular, light emitted from an LED or a like element disposed in the power supply section 31 of FIG. 2 may be propagated to the distal end of the elongated member 11 through a light guide configured of glass, plastic or a like material such that the light is irradiated from the distal end of the elongated member 11.

The balloon 12 is expanded when liquid or gas is supplied thereto from the pressure supplying section 30 connected to the pressure supply connection portion 15b. Consequently, if the balloon 12 is expanded in a state in which the balloon 12 is inserted in a constriction portion of a natural ostium, then the constriction portion of the natural ostium can be expanded.

The CT scanner 300 is an apparatus which can acquire a three-dimensional CT image which is a three-dimensional image of the inside of the human body by scanning an object using a radiation and processing data obtained by scanning the object using a computer.

The inputting section 100 receives the two-dimensional image picked up by the camera provided at the distal end of the catheter 200 inserted in the body of the subject from the catheter 200. The inputting section 100 transmits the received two-dimensional image from the catheter 200 to the extraction section 120 and the display section 140.

Further, the inputting section 100 receives the three-dimensional CT image of a subject same as the subject whose two-dimensional image is picked up from the CT scanner 300. The inputting section 100 transmits the inputted three-dimensional CT image to the recording section 110 so as to be recorded by the recording section 110.

The recording section 110 stores the three-dimensional CT image received from the inputting section 100 and transmits the stored three-dimensional CT image to the detection section 130. It is to be noted that preferably a three-dimensional CT image acquired before insertion of the catheter 200 into the subject is stored in advance into the recording section 110.

The extraction section 120 extracts a contour shape of at least one of organs of the subject represented by the two-dimensional image received from the inputting section 100. The extraction of the contour shape of an organ of the subject represented by a two-dimensional image can be carried out using a known edge extraction filter which extracts the contour shape focusing on a sudden change of the intensity of pixels on the contour. For example, a differential filter, a Prewitt filter or a Sobel filter can be used.

It is to be noted that the extracted contour shape of the organ may partly include a contour shape by a boundary line generated by an overlap of organs displayed on a two-dimensional image.

The extraction section 120 transmits the contour shape (hereinafter referred to as “two-dimensional contour shape”) of the organ extracted from the two-dimensional image to the detection section 130.

The detection section 130 detects, in the three-dimensional CT image received from the recording section 110, a cross section including the contour shape which at least partly coincides with the two-dimensional contour shape received from the extraction section 120. The detection section 130 three-dimensionally moves the cross section including the two-dimensional contour shape received from the extraction section 120 on a three-dimensional coordinate of the three-dimensional CT image. Further, the detection section 130 enlarges or reduces the two-dimensional contour shape on the cross section to carry out collation therebetween. By the collation, the detection section 130 can detect the cross section of the three-dimensional CT image having the contour shape which at least partly coincides with the two-dimensional contour shape. It is to be noted that, thereupon, the cross section having the contour shape which at least partly coincides with the two-dimensional contour shape in a three-dimensional contour shape extracted by carrying out filtering of the three-dimensional CT image by an edge extraction filter may be detected.

The two-dimensional contour shape extracted by the extraction section 120 may possibly include the contour shape by a boundary line which appears by an overlap of organs displayed on the two-dimensional image with each other. In this instance, contours of a plurality of organs may possibly be included in one extracted closed two-dimensional contour shape. Therefore, there is the possibility that the cross section including the contour shape coincident with the two-dimensional contour shape may not be able to detect in the three-dimensional CT image. However, if a portion other than a boundary line which appears by an overlap of organs in the contour shape of the organs with each other exists in the two-dimensional contour shape, then the contour shape of the portion exists in the three-dimensional CT image. Accordingly, the detection section 130 detects, in a three-dimensional CT image, the cross section including the contour shape which at least partly coincides with the contour shape of an organ in a two-dimensional image.

The detection section 130 transmits a portion of a three-dimensional CT image which includes the cross section detected thereby to the display section 140.

The display section 140 displays the two-dimensional image received from the inputting section 100 and the three-dimensional CT image received from the detection section 130 at the same time. The three-dimensional CT image includes the organ having the cross section including the contour shape which at least partly coincides with the contour shape of the organ displayed on the two-dimensional image. In other words, the display section 140 displays the two-dimensional image and the three-dimensional CT image which include the same organ at the same time.

This allows stereoscopic grasping of the organ and its surrounding region of the two-dimensional image through the three-dimensional CT image and further facilitates grasping of the position of the natural ostium whose confirmation is difficult on the two-dimensional image from the image pickup element of the balloon catheter.

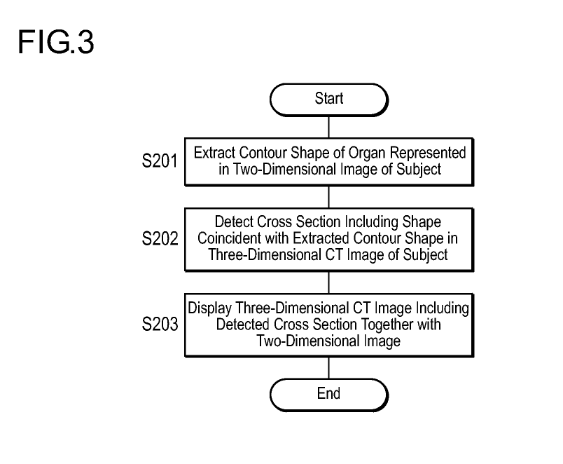

FIG. 3 illustrates a flow chart of a catheter insertion supporting method according to the present embodiment. Processing of the present flow chart can be carried out by the catheter insertion supporting system according to the present embodiment.

The catheter insertion supporting system 1 extracts the contour shape of at least one of organs of the subject represented on the two-dimensional image by the catheter 200 at step S201.

Then at step S202, the catheter insertion supporting system 1 detects, in the three-dimensional CT image picked up by the CT scanner 300, the cross section including the contour shape which at least partly coincides with the contour shape extracted at step S201.

Thereafter, the catheter insertion supporting system 1 displays the three-dimensional CT image at the portion including the organ having the cross section detected at step S202 together with the two-dimensional image at step S203.

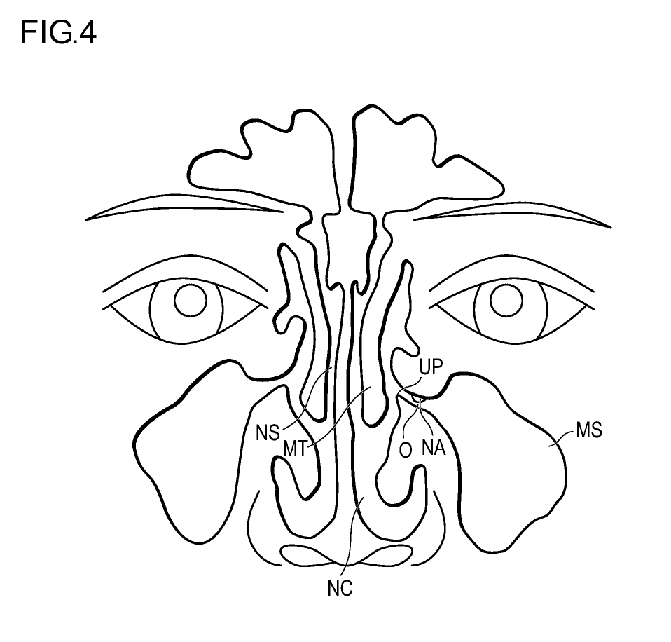

FIG. 4 is a view showing, in a simplified form, an anatomical structure of the inside of the nose of a patient having a constriction portion NA formed in the natural ostium O communicating with a maxillary sinus MS which is one of the paranasal sinuses.

The catheter 200 is introduced to the interior of the nasal cavity NC. The natural ostium O communicating with the maxillary sinus MS may be partly covered with the middle turbinate MT or the uncinate process UP. Besides, in order to confirm the position of the natural ostium O, it is necessary to first advance the catheter 200 by a predetermined distance in an approach path extending along the nasal cavity NC and then direct the field of view of the catheter 200 sideward or to the rear side. Therefore, it is sometimes difficult to find out the natural ostium O by the catheter 200 where the catheter 200 has a narrow field of view.

However, according to the present embodiment, the organ and its peripheral region displayed in the two-dimensional image of the catheter 200 can be grasped stereoscopically through the three-dimensional CT image. Therefore, the position of the natural ostium O whose confirmation is difficult in the two-dimensional image of the balloon catheter 200 can be grasped readily.

It is to be noted that, at step S202 of FIG. 3, alternatively the range of the three-dimensional CT image may be limited such that the cross section including the contour shape which at least partly coincides with the contour shape extracted at step S201 is detected within the limited range. By limiting the range of the three-dimensional CT image which is a target of detection, the arithmetic operation time required for the detection can be reduced.

For example, the three-dimensional CT image of the target of detection may be limited to a range in which the signal intensity at each coordinate configuring the three-dimensional CT image does not exceed a threshold value determined in advance and a peripheral range around the range. The range into which the catheter 200 can be inserted is a cavity in a bone such as the nasal cavity, and the signal intensity in the cavity in the bone is comparatively low in the three-dimensional CT image. Accordingly, the range in which there is the possibility that at least part of the contour shape of the organ represented in the two-dimensional image by the catheter 200 may coincide is limited to a region of the cavity in the bone and its peripheral region. Therefore, by the limitation of the three-dimensional CT image described above, the detection time can be reduced without having an influence on the detection result at step S201.

Alternatively, the three-dimensional CT image of the detection target may be limited to a visible range of the catheter 200. The visible range of the catheter 200 depends upon the insertion distance of the catheter 200 in the human body, the field of view and the direction of the image pickup element. Here, the field of view of the image pickup element is a range within which an image is picked up as the two-dimensional image by the image pickup element.

The insertion distance of the catheter 200 in the human body can be measured using light, magnetism, an ultrasonic wave or the like. For example, a sensor, an oscillator or a detection target which can be detected by a sensor is provided at at least one of an insertion opening for the catheter 200 into the human body such as the nostril and at an arbitrary location of the catheter 200 located outside the human body such as, for example, the hub 15.

For example, a sensor having a coil built therein is installed at the insertion opening for the catheter 200 into the human body and current is supplied to the coil so that a high frequency magnetic field is generated. If a metal element which is a target of detection installed on the hub 15 of the catheter 200 comes near to the sensor, then induced current by electromagnetic induction flows through the coil of the sensor in response to the degree of the approach of the metal element and varies the impedance of the coil. Accordingly, by detecting the variation of the impedance of the coil, the insertion distance of the catheter 200 into the human body can be measured.

As an alternative, a capacitor wherein an insulator is sandwiched between electrodes is installed at the insertion opening for the catheter 200 into the human body. If a target of detection installed on the hub 15 of the catheter 200 comes near to the capacitor, then the capacitance of the capacitor varies in response to the degree by which the detection target comes near. Accordingly, by detecting the variation of the capacitance of the capacitor, the insertion distance of the catheter 200 into the human body can be measured.

As another alternative, an ultrasonic vibration element and an ultrasonic wave detector are installed at the insertion opening for the catheter 200 into the human body. The ultrasonic vibration element generates an ultrasonic wave, and the ultrasonic wave detector detects the ultrasonic wave reflected by a reflecting plate installed on the hub 15 of the catheter 200. By measuring the variation of feedback time after the ultrasonic vibration element generates an ultrasonic wave until the ultrasonic wave reflected by the reflecting plate is detected by the ultrasonic wave detector, the insertion distance of the catheter 200 into the human body can be measured.

As a further alternative, the insertion distance of the catheter 200 into the human body can be measured by measuring the variation of feedback time of an electromagnetic wave generated at the insertion opening for the catheter 200 into the human body. In this instance, the variation of the feedback time of the electromagnetic wave is measured until the electromagnetic wave generated at the insertion opening for the catheter 200 into the human body is reflected by a reflecting plate installed on the hub 15 of the catheter 200 and detected at the insertion opening for the catheter 200 into the human body.

As a still further alternative, the insertion distance of the catheter 200 into the human body may be measured by measuring the period of time after infrared rays are generated by an infrared ray generation element installed at the insertion opening for the catheter 200 into the human body until the infrared rays are electrically detected by an infrared sensor installed on the hub 15 of the catheter 200.

In addition to the method described above, such a method may be adopted as to compare the two-dimensional image by the catheter 200 between successive frames to detect a variation such as a forward or backward movement or a rotational movement, or the direction and the amount of the variation.

The field of view of the catheter 200 is determined in advance based on the specifications of the image pickup element of the catheter 200. The field of view of the catheter 200 depends upon the view angle, focal length and so forth of the camera.

The direction of the image pickup element of the catheter 200 can be specified by detecting a posture of the catheter 200, for example, in such a case that the distal end portion of the catheter 200 at which the image pickup element is provided can be bent by a bending manipulation. Here, the posture of the catheter 200 signifies the appearance of the catheter 200 at each step where the distal end of the catheter 200 bends in a stepwise fashion. The posture of the catheter 200 can be detected based on an adjustment amount for changing the posture of the catheter 200.

Further, at step S202 of FIG. 3, when the cross section including the contour shape which at least partially coincides with the contour shape extracted from the two-dimensional image is to be detected, the arithmetic operation time required for the detection can be reduced by gradually decreasing the step size of the cross section of the three-dimensional CT image of a target of detection.

For example, the cross sections of the three-dimensional CT image are cut out first at distances of 5 mm to narrow down the range which exhibits a high degree of similarly to the contour shape extracted from the two-dimensional image. Then, within the narrowed down range, cross sections of the three-dimensional CT image are cut out at distances of 1 mm to further narrow down the range. Finally, by cutting out cross sections of the three-dimensional CT image at distances of 0.1 mm, the cross section including a contour shape which coincides with the contour shape extracted from the two-dimensional image can be detected.

It is to be noted that it is possible to utilize the present embodiment to carry out calibration for adapting the coordinate system of the three-dimensional CT image and the position of the image pickup element of the catheter to each other.

In particular, a relative positional relationship between the organ having the cross section detected in the three-dimensional CT image at step S202 and the image pickup element of the catheter 200 is calculated based on the two-dimensional image and the specifications of the field of view of the image pickup element. Then, based on the calculated positional relationship and the position of the organ in the three-dimensional CT image, the position of the image pickup element of the catheter 200 on the coordinate system of the three-dimensional CT image is specified. Consequently, calibration for adapting the coordinate system of the three-dimensional CT image and the position of the image pickup element of the catheter 200 to each other can be carried out.

With such calibration, it is possible to implement reduction of the cost because the necessity for hardware for detecting the position of the image pickup element is eliminated. Further, also within a period within which inspection or treatment by insertion of the catheter 200 is being carried out, the calibration can be carried out suitably and easily.

The position of the image pickup element of the catheter 200 on the coordinate system of the three-dimensional CT image specified by the calibration can be displayed for specification, for example, by displaying a marking at the position of the image pickup element in the three-dimensional CT image. Consequently, a doctor and so forth can carry out insertion of the catheter 200 and inspection and treatment by the catheter 200 while suitably confirming the position of the catheter 200 in the three-dimensional CT image.

Although the catheter insertion supporting system, catheter insertion supporting method, the computer readable storage medium stored with the program for the catheter insertion supporting system, and calibration method according to the first embodiment of the present invention have been described above, the present embodiment exhibits the following effect.

In particular, the contour shape of an organ is extracted from the two-dimensional image from the image pickup element of the balloon catheter, and the cross section including the contour shape which coincides with the extracted contour shape is detected in the three-dimensional CT image. Then, the three-dimensional CT image in a region which includes the organ having the detected cross section is displayed together with the two-dimensional image from the image pickup element. This makes it possible to stereographically grasp the organ and its peripheral region displayed in the two-dimensional image through the three-dimensional CT image and easily grasp the position of the natural ostium whose confirmation is difficult in the two-dimensional image from the image pickup element of the balloon catheter.

### Second Embodiment

A catheter insertion supporting system, a catheter insertion supporting method, a computer readable storage medium stored with a program for the catheter insertion supporting system, and a calibration method according to a second embodiment of the present invention are described below.

The present embodiment is different from the first embodiment in that, in the present embodiment, the correspondence relationship of organs represented on both of the three-dimensional CT image and the two-dimensional image is specified and the organs are displayed in different colors. The other points of the present embodiment are similar to those of the first embodiment, and therefore, an overlapping description of them is omitted or simplified herein.

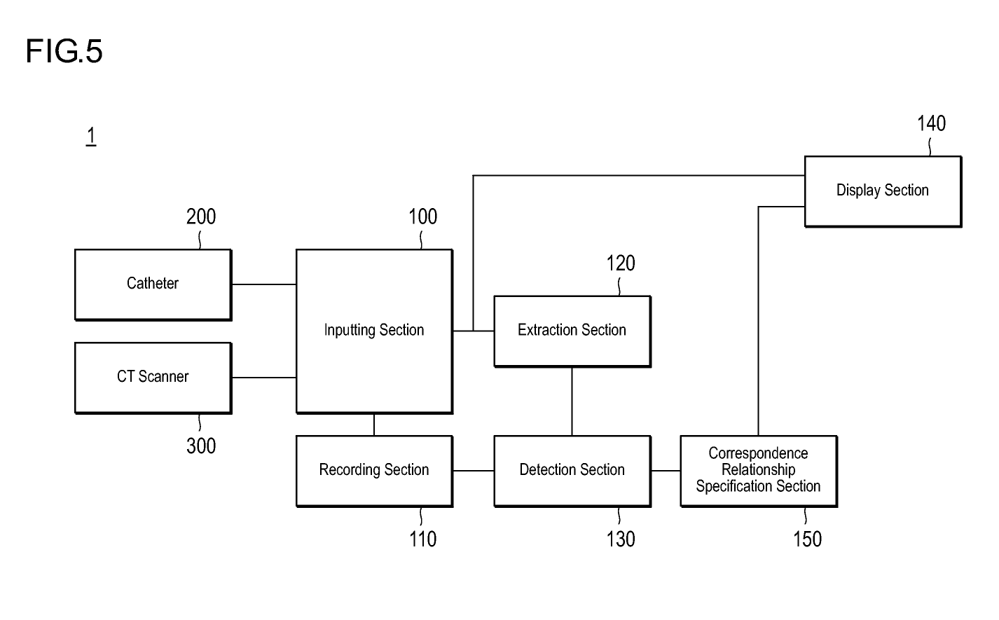

FIG. 5 is a block diagram depicting a configuration of the catheter insertion supporting system according to the second embodiment of the present invention.

Referring to FIG. 5, the catheter insertion supporting system 1 according to the present embodiment includes an inputting section 100, a recording section 110, an extraction section 120, a detection section 130, a correspondence relationship specification section 150, a display section 140, a catheter 200 and a CT scanner 300. The inputting section 100, the recording section 110, the extraction section 120, the detection section 130, the correspondence relationship specification section 150 and the display section 140 can be configured of a computer and a program.

The detection section 130 detects, in the three-dimensional CT image received from the recording section 110, the cross section including the contour shape which at least partly coincides with the two-dimensional contour shape received from the extraction section 120. Thereupon, the detection section 130 detects, in the three-dimensional contour shape extracted by carrying out filtering by an edge extraction filter for the three-dimensional CT image (hereinafter referred to as “three-dimensional contour shape”), the cross section having the contour shape which at least partly coincides with the two-dimensional contour shape.

The extraction of the three-dimensional contour shape can be carried out using a known edge extraction filter. For example, a differential filter, a Prewitt filter or a Sobel filter can be used.

The correspondence relationship specification section 150 receives the two-dimensional image, the two-dimensional contour shape, a portion of the three-dimensional CT image which includes the cross section detected by the detection section 130, and the three-dimensional contour shape of the three-dimensional CT image from the detection section 130.

The correspondence relationship specification section 150 adapts the two-dimensional contour shape and the three-dimensional contour shape which has the cross section which at least partly coincides with the two-dimensional contour shape. Consequently, the correspondence relationship specification section 150 specifies the correspondence relationship between organs represented in the two-dimensional image and organs in the three-dimensional CT image. In other words, the correspondence relationship specification section 150 specifies the correspondence relationship between the organs in a unit of a contour shape of the organ displayed in the three-dimensional CT image and the two-dimensional image.

The correspondence relationship specification section 150 transmits the correspondence relationship of organs represented in the three-dimensional CT image and the two-dimensional image and the portion of the three-dimensional CT image which includes the cross section detected by the detection section 130 to the display section 140.

The display section 140 displays the correspondence relationship between organs in the three-dimensional CT image and organs represented in the two-dimensional image by color coding in the three-dimensional CT image and the two-dimensional image based on the correspondence relationship received from the correspondence relationship specification section 150. Thereupon, the display section 140 carries out color coding in a unit of the contour shape of the organ displayed in the three-dimensional CT image and the two-dimensional image to indicate the correspondence relationship between the organs in the three-dimensional CT image and the organs in the two-dimensional image.

It is to be noted that the display section 140 may display the correspondence relationship between organs in the three-dimensional CT image and organs represented in the two-dimensional image by a method different from the color coding. For example, the correspondence relationship between organs in the three-dimensional CT image and organs represented in the two-dimensional image may be displayed using different types of slanting lines to be applied to the individually corresponding organs or using different numerals to be applied to the individually corresponding organs.

By displaying organs represented in both of the three-dimensional CT image and the two-dimensional image using color coding for the individual organs in this manner, the position of the catheter at present in the nasal cavity based on the two-dimensional image can be grasped readily by referring to the color and the shape of the organs represented simultaneously in the three-dimensional CT image.

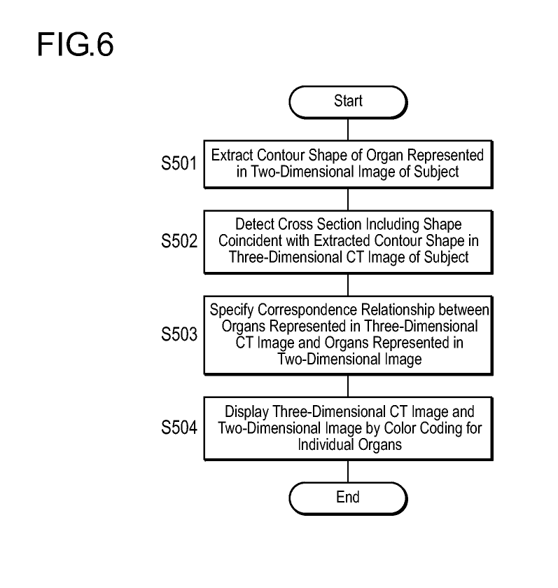

FIG. 6 is a view illustrating a flow chart of the catheter insertion supporting method according to the present embodiment. Processing of the present flow chart can be carried out by the catheter insertion supporting system according to the present embodiment.

The catheter insertion supporting system 1 extracts the contour shape of at least one of organs of a subject represented in the catheter two-dimensional image at step S501.

Then at step S502, the catheter insertion supporting system 1 detects, in the three-dimensional CT image picked up by the CT scanner 300, the cross section including the contour shape which at least partly coincides with the contour shape extracted at step S501.

The catheter insertion supporting system 1 adapts the organ having the cross section detected at step S502 and the contour shape of the two-dimensional image which at least partly coincides with the contour shape included in the cross section to each other. The catheter insertion supporting system 1 thereby specifies the correspondence relationship between the organs in the three-dimensional CT image and the organs represented in the two-dimensional image at step S503.

Then at step S504, the catheter insertion supporting system 1 displays the correspondence relationship between the two-dimensional image and the three-dimensional image by color coding for the individual organs having the correspondence relationship therebetween based on the correspondence relationship of the organs specified at step S503.

The catheter insertion supporting system, catheter insertion supporting method, the computer readable storage medium stored with the program for the catheter insertion supporting system, and calibration method according to the second embodiment of the present invention are described above. The present embodiment exhibits the following effect in addition to the effect exhibited by the first embodiment.

The organs represented on both of the three-dimensional CT image and the two-dimensional image are displayed by color coding for each same organ. Therefore, the position of the catheter at present in the nasal cavity based on the two-dimensional image can be grasped readily by referring to the color and the shape of the organ represented simultaneously in the three-dimensional CT image.

Although the catheter insertion supporting system, catheter insertion supporting method, the computer readable storage medium stored with the program for the catheter insertion supporting system, and calibration method according to the first and second embodiments of the present invention are described above, the present invention is not limited to the embodiments described above.

For example, in the embodiment described above, the catheter insertion supporting system adapted to a balloon catheter for treating a constriction of the natural ostium is taken as an example and described. However, the expansion structure is not limited to the balloon, but the catheter may have some other expansion structure. Alternatively, the catheter may be a drug release, a stent or an ablation device or may have an ablation function. Also the insertion location is not limited to the nose but may be an ear, a throat or some other body cavity. Further, the present invention may be applied also to stereoscopically grasp the position of a tumor which is difficult to confirm in the two-dimensional image by the camera provided on the catheter, through the three-dimensional CT image and carry out catheter treatment of the tumor.

Further, if the three-dimensional CT image includes a plurality of candidates for the organ having the cross section including the contour shape which at least partly coincides with the extracted contour shape, then the three-dimensional CT image selected from among the plural candidates by the user may be displayed. Alternatively, the three-dimensional CT image of one of the candidates which exhibits a higher rate at which the extracted contour shape and the contour shape on the cross section coincide with each other may be displayed.

### Third Embodiment

In the following, a catheter insertion supporting system, a catheter insertion supporting method, a computer readable storage medium stored with the program for the catheter insertion supporting system, and a calibration method according to a third embodiment of the present invention are described in detail. It is to be noted that description of the contents which are common to those of the first embodiment is omitted herein to avoid redundancy.

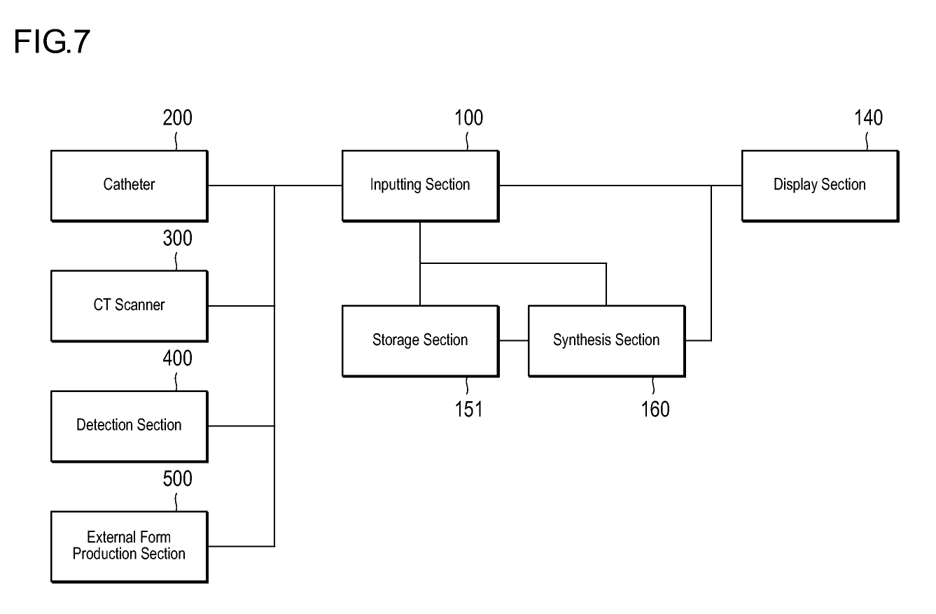

FIG. 7 is a block diagram depicting a configuration of the catheter insertion supporting system according to the third embodiment of the present invention.

Referring to FIG. 7, the catheter insertion supporting system 1 according to the present embodiment includes an inputting section 100, a storage section 151, a synthesis section 160, a display section 140, a catheter 200, a CT scanner 300, a detection section 400, and an external form production section 500. The inputting section 100, the storage section 151, the synthesis section 160 and the display section 140 can be configured of a computer and a program.

The detection section 400 detects a position, a direction and a posture, in the human body, of the catheter 200 inserted in the body of a subject.

It is to be noted that, when the position and the direction of the catheter 200 in the body are detected by the detection section 400, a three-dimensional coordinate system is set to the body of the subject. The setting of the three-dimensional coordinate system to the body of the subject can be carried out based on a three-dimensional positional relationship of a plurality of locations of the catheter 200 when the catheter 200 is contacted with the plural locations of the surface of the body of the subject and the detection section 400 detects the position of the catheter 200 at each contacting position. Further, calibration of adapting the three-dimensional coordinate system set to the body of the subject and a three-dimensional coordinate system of a three-dimensional CT image of the subject acquired by the CT scanner 300 to each other is carried out. Here, when the catheter 200 is brought into contact with the body of the subject, there is the possibility that the image pickup element of the catheter 200 may be soiled. However, such soiling can be prevented by temporarily covering the image pickup element with a cap.

Also it is possible to carry out the calibration making use of the first embodiment described hereinabove. In particular, (a) at steps S201 and S202 of FIG. 3, an image of a characteristic organ of the living body of the subject such as, for example, the nasal cavity, inferior turbinate, nasal septum or middle turbinate is picked up by the image pickup element of the catheter 200. Then, the characteristic organ is displayed in a two-dimensional image. Then, the characteristic organ in the displayed two-dimensional image is adapted to a three-dimensional CT image. This can be carried out readily by detecting a cross section whose contour shape partially coincides with a contour shape extracted from the displayed two-dimensional image in the three-dimensional CT image acquired in advance and then displaying the characteristic organ having the cross section in the three-dimensional CT image. (b) The position of the catheter 200 when the cross section including the contour shape which at least partially coincides with the contour shape extracted from the two-dimensional image at step S202 is detected in the three-dimensional CT image is detected by the detection section 400. (c) The relative positional relationship between the characteristic organ having the cross section detected in the three-dimensional CT image at step S202 and the image pickup element of the catheter 200 is calculated based on the two-dimensional image and the specifications of the field of view of the image pickup element. (d) The position of the catheter 200 detected by the detection section 400 in the paragraph (b) above is corrected to the position of the characteristic organ by utilizing the relative positional relationship between the characteristic organ and the image pickup element of the catheter 200 calculated in the paragraph (c) above. (e) The operations in the paragraphs (a) to (d) are carried out by a plural number of times to set the three-dimensional coordinate system of the detection section 400 with respect to the body of the subject based on the three-dimensional positional relationship of the positions of a plurality of characteristic organs detected. (f) A calibration of adapting the three-dimensional coordinate system of the detection section 400 set to the body of the subject and the three-dimensional coordinate system of the three-dimensional CT image of the subject acquired by the CT scanner 300 to each other using the positions of the characteristic organs which are same in the coordinate systems as indexes is carried out.

Preferably, the calibration is suitably carried out also while inspection and treatment by the catheter 200 are being carried out in order to maintain the accuracy of the calibration. Such calibration which makes use of the first embodiment as described above can be carried out readily and flexibly also while inspection and treatment by the catheter 200 are being carried out. Besides, since there is no necessity to contact the catheter 200 with the living body of the subject, soiling to the image pickup element by contact of the image pickup element of the catheter 200 with the living body can be prevented.

The detection section 400 can detect the position of the catheter 200 as a coordinate on the three-dimensional coordinate system set to the body of the subject. Preferably, the detection section 400 detects the position of the distal end of the catheter 200. The position of the catheter 200 can be detected, for example, by reception of responding light based on excitation by excitation light radiated from the distal end of the catheter 200. Alternatively, the position of the catheter 200 may be detected utilizing a response when an ultrasonic wave is applied to the body of the subject. Alternatively, the position of the catheter 200 may be calculated based on an angle of the catheter 200 detected by one of a plurality of angle sensors provided between one end of the catheter 200 and a fixture. It is to be noted that the fixture may be provided on the hub 15. Otherwise, the position of the catheter 200 may be detected using a magnetic sensor provided at the distal end of the catheter 200. Alternatively, the position of the distal end of the catheter 200 may not be detected directly, but the positional relationship between the distal end of the catheter 200 and the hub 15 by the angle sensor or the like while the position, posture and direction of the hub 15 is detected and specified by a different sensor. It is to be noted that, when the position of the catheter 200 is detected as a coordinate on the three-dimensional coordinate system set to the body of the subject, the range of the coordinate system may be limited such that the position of the catheter 200 is detected within the limited range. This can reduce the time required for the detection of the position of the catheter 200. For example, the insertion distance of the catheter 200 from the insertion opening such as, for example, the nostril is detected such that the range of the coordinate system is limited to the range of the insertion distance from the insertion opening to detect the position of the catheter 200. Here, the insertion distance of the catheter 200 can be measured by a method similar to that in the first embodiment described hereinabove.

The detection section 400 can detect the direction of the catheter 200 as a vector toward the distal end of the catheter 200 in the longitudinal direction. The direction of the catheter 200 can be detected, for example, by reception of responding light based on excitation by excitation light radiated from the distal end and the proximal end of the catheter 200. Alternatively, the direction of the catheter 200 may be detected by utilization of a response when an ultrasonic wave is applied to the body of the subject. Alternatively, the direction of the catheter 200 may be detected using a magnetic sensor provided at the distal end and the proximal end of the catheter.

The detection section 400 detects the posture of the catheter 200 based on the adjustment amount of the curve operation portion 17 for changing the posture of the catheter 200. For example, when numerical value data is inputted to the catheter 200 to change the posture of the catheter 200, the posture of the catheter 200 can be detected based on the numerical data. On the other hand, where the curve operation portion 17 of the catheter 200 is configured such that the posture of the catheter 200 is changed by rotating a rotating knob, the rotational angle of the knob may be detected by an angle sensor such that the posture of the catheter 200 is detected based on the displacement amount of the detected rotational angle.

The detection section 400 transmits the detected position, direction and posture of the catheter 200 in the body to the inputting section 100.

The external form production section 500 produces the three-dimensional image of an external form of the catheter 200. It is to be noted that the external form production section 500 produces the three-dimensional image of the external form of the catheter 200 with regard to each of postures which the catheter 200 assumes.

The external form production section 500 can be configured, for example, of a stereo camera. Here, the stereo camera signifies an image pickup apparatus which picks up a three-dimensional image of an external form of an object by the principle of triangulation. It is to be noted that the external form production section 500 may produce the three-dimensional image of the external form of the catheter 200 based on design data of the catheter 200.

The inputting section 100 receives the two-dimensional image picked up by the image pickup element provided at the distal end of the catheter 200 inserted in the body of a subject from the catheter 200. The inputting section 100 transmits the received two-dimensional image to the display section 140.

The inputting section 100 further receives the three-dimensional CT image of the subject same as the subject whose two-dimensional image is picked up from the CT scanner 300. The inputting section 100 stores the received three-dimensional CT image into the storage section 151.

The inputting section 100 further receives the position, the direction and the posture of the catheter 200 in the body of a subject detected by the detection section 400. The inputting section 100 transmits the received position, direction and posture of the catheter 200 in the body of the subject to the storage section 151 so as to be stored.

The inputting section 100 further receives the three-dimensional image of the external form of the catheter 200 produced by the external form production section 500. The inputting section 100 transmits the received three-dimensional image of the external form of the catheter 200 to the storage section 151 so as to be stored.

The storage section 151 stores the three-dimensional CT image and the position, the direction and the posture of the catheter 200 in the body of the subject received from the inputting section 100. The storage section 151 may store the three-dimensional image of the external form of the catheter 200 produced in advance by the external form production section 500. Further, the storage section 151 transmits the three-dimensional CT image, the position, the direction and the posture of the catheter 200 in the body of the subject and the three-dimensional image of the external form of the catheter 200 stored therein to the synthesis section 160 in accordance with a request of the synthesis section 160.

It is to be noted that, where a marking is applied as a place to be noticed to a particular place of a three-dimensional CT image by the user, the storage section 151 stores the three-dimensional CT image having the marking applied thereto.

The synthesis section 160 issues a request for the three-dimensional CT images to the storage section 151 and receives them from the storage section 151. Further, the synthesis section 160 issues a request for the position, the direction and the posture of the catheter 200 in the body detected by the detection section 400 to the storage section 151 and receives them from the storage section 151. Where the three-dimensional image of the external form of the catheter 200 acquired or produced in advance is stored in the storage section 151, the synthesis section 160 may issue a request for the three-dimensional image of the external form of the catheter 200 to the storage section 151.

Then, the synthesis section 160 combines the three-dimensional image of the external form of the catheter 200 into the three-dimensional CT image so that the position, the direction and the posture of the catheter 200 in the body and the position, the direction and the posture of the three-dimensional image of the external form of the catheter 200 in the three-dimensional CT image may coincide with each other. Consequently, the three-dimensional image of the external form of the catheter 200 and the three-dimensional CT image are synthesized. Thereupon, in order to adapt the postures to each other, any one of the three-dimensional images produced by the external form production section 500 and stored in the storage section 151 regarding the postures which the catheter 200 can take can be selected. It is to be noted that the synthesis section 160 may produce the three-dimensional image of the external form of the catheter 200 to be combined with the three-dimensional CT image by CG (Computer Graphics) rendering based on the posture of the catheter 200 in the body detected by the detection section 400.

It is to be noted that, where the three-dimensional CT image has the marking applied thereto by the user, the synthesis section 160 synthesizes the three-dimensional image of the external form of the catheter 200 and the three-dimensional CT image while the marking is kept applied.

The display section 140 displays the three-dimensional CT image on which the three-dimensional image of the external form of the catheter 200 is synthesized and the two-dimensional image picked up by the image pickup element of the catheter 200 at the same time. Thereupon, the display section 140 displays the three-dimensional CT image synthesized by the synthesis section 160 so as to be rotatable by rotating an arbitrary one of coordinate axes of the coordinate system of the three-dimensional CT image. The user can rotate and display the three-dimensional CT image to a predetermined direction by designating a coordinate axis to be rotated and a rotational angle.

Accordingly, the user can simultaneously confirm both of the three-dimensional image of the external form of the catheter 200 in the three-dimensional CT image in which the position, the direction and the posture of the catheter 200 inserted in the body of the subject are displayed and the two-dimensional image of the inside of the body by the image pickup element provided at the distal end of the catheter 200. Consequently, the user can stereoscopically grasp the positional relationship between the position of the distal end of the catheter 200 and the natural ostium and can stereoscopically grasp organs and the periphery of the organs displayed on the two-dimensional image from the three-dimensional CT image.

Further, the user can grasp the positional relationship between the distal end of the catheter 200 inserted in the body and organs in the body with a higher degree of definiteness by three-dimensionally rotating the three-dimensional CT image on which the external form of the catheter 200 in the body is synthesized.

Further, the user can grasp the positional relationship between the distal end of the catheter 200 in the body and the marking applied in advance to the three-dimensional CT image with a higher degree of definiteness by causing the marking to be displayed in the three-dimensional CT image on which the external form of the catheter 200 in the body is synthesized.

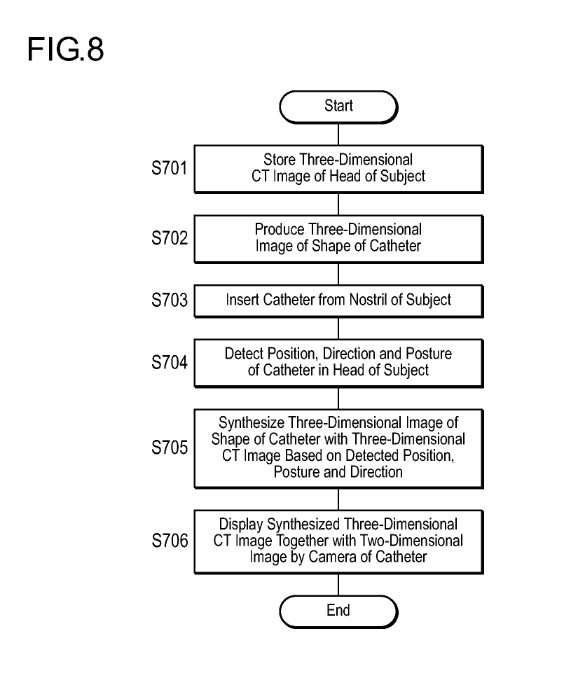

FIG. 8 is a view depicting a flow chart of the catheter insertion supporting method according to the present embodiment. Processes of the present flow chart can be carried out by the catheter insertion supporting system 1 according to the present embodiment.

First at step S701, the storage section 151 stores the three-dimensional CT image of the head of the subject acquired from the CT scanner 300.

Then at step S702, the external form production section 500 picks up an image of the catheter 200 to produce the three-dimensional image of a shape of the catheter 200. At this time, the external form production section 500 produces the three-dimensional image of the external form of the catheter 200 with regard to each of various postures which are taken by the catheter 200.

At step S703, the user would insert the catheter 200 from the nostril of the subject.

At step S704, the detection section 400 detects the position, the direction and the posture of the catheter 200 with respect to the head of the subject.

At step S705, the synthesis section 160 synthesizes the three-dimensional image of the shape of the catheter 200 with the three-dimensional CT image based on the detected position, posture and direction of the catheter 200 with respect to the head of the subject. In particular, the synthesis section 160 combines the three-dimensional image of the external form of the catheter 200 into the three-dimensional CT image such that the position, the direction and the posture of the catheter 200 with respect to the head of the subject and the position, the direction and the posture of the three-dimensional image of the external form of the catheter 200 in the three-dimensional CT image may coincide with each other, respectively. Consequently, the three-dimensional image of the external form of the catheter 200 and the three-dimensional CT image are synthesized with each other. The three-dimensional image of the shape of the catheter 200 combined with the three-dimensional CT image is the three-dimensional image produced at step S702 by the external form production section 500. However, the three-dimensional image may otherwise be the three-dimensional image rendered by the synthesis section 160 based on the posture of the catheter 200 with respect to the head of the subject detected at step S704.

Then at step S706, the display section 140 displays the three-dimensional CT image on which the three-dimensional image of the external form of the catheter 200 is synthesized by the synthesis section 160 together with the two-dimensional image from the image pickup element of the catheter 200. Thereupon, the display section 140 displays the three-dimensional CT image rotatably in three dimensions.

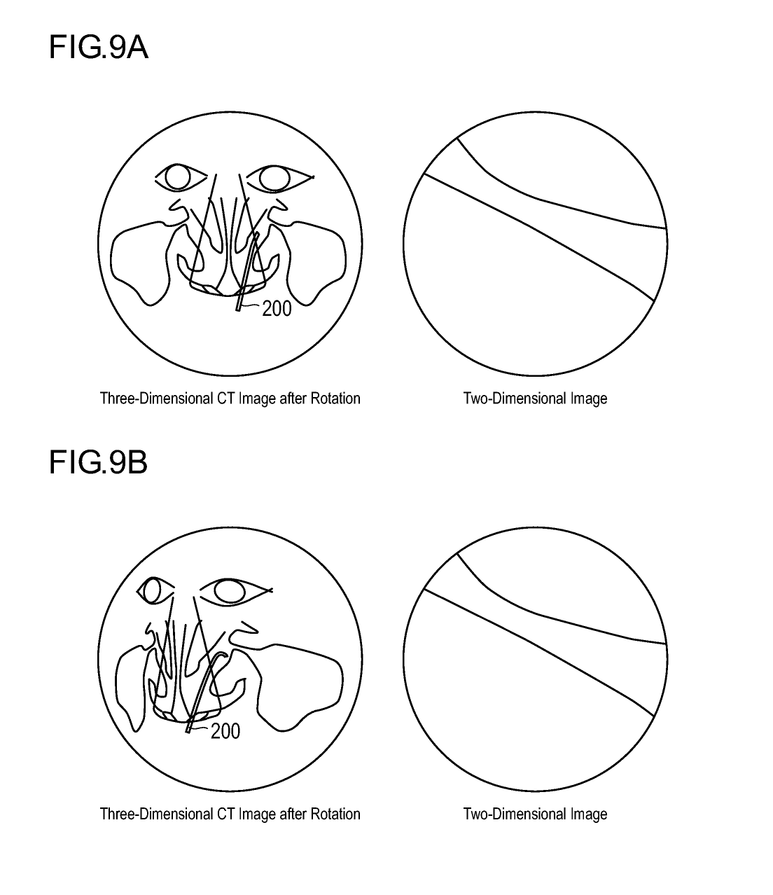

FIGS. 9A and 9B illustrate the three-dimensional CT image on which the three-dimensional image of the external form of the catheter is synthesized in a state in which the three-dimensional CT image is displayed together with the two-dimensional image from the image pickup element of the catheter. Particularly, FIG. 9A depicts the three-dimensional CT image before rotation in a state in which it is displayed together with the two-dimensional image from the image pickup element of the catheter. Meanwhile, FIG. 9B depicts the three-dimensional CT image after rotation in a state in which it is displayed together with the two-dimensional image from the image pickup element of the catheter.

As seen from FIGS. 9A and 9B, the user can grasp the positional relationship between the distal end of the catheter 200 inserted in the body and organs in the body with a higher degree of definiteness by three-dimensionally rotating the three-dimensional CT image on which the three-dimensional image of an external form of the catheter 200 in the body is synthesized.

The catheter insertion supporting system, catheter insertion supporting method, computer readable storage medium stored with the program for the catheter insertion supporting system, and calibration method according to the third embodiment of the present invention are described above. The present embodiment exhibits the following effects.

After the three-dimensional image of the subject is stored, the position and the direction of the catheter in the body are detected. Then, the three-dimensional image of the external form of the catheter is combined into and synthesized with the three-dimensional CT image such that the position and the direction of the external form of the catheter of the three-dimensional image in the three-dimensional CT image may coincide with the detected position and direction of the catheter in the body, respectively. The synthesized image is displayed together with the two-dimensional image from the camera of the catheter. Consequently, the positional relationship between the position of the distal end of the catheter and the natural ostium can be grasped stereoscopically. Further, organs and its peripheral elements displayed on the two-dimensional image can be stereoscopically grasped from the three-dimensional CT image.

Further, it is possible to grasp the positional relationship between the distal end of the catheter inserted in the body and organs in the body with a higher degree of definiteness by rotatably displaying the three-dimensional CT image on which the three-dimensional image of the external form of the catheter in the body is synthesized.

Further, it is possible to grasp the positional relationship between the distal end of the catheter in the body and the marking applied in advance to the three-dimensional CT image with a higher degree of definiteness by causing the marking to be displayed in the three-dimensional CT image on which the three-dimensional image of the external form of the catheter in the body is synthesized.

Although the catheter insertion supporting system, catheter insertion supporting method, computer readable storage medium stored with the program for the catheter insertion supporting system, and calibration method according to the third embodiment of the present invention are described above, the present invention is not limited to the embodiment described above.

For example, in the embodiment described above, the catheter insertion supporting system applied to the balloon catheter for treating a constriction of the natural ostium is taken as an example. However, the present invention can be applied to stereoscopically grasp the positional relationship between the position of a tumor and the catheter, which is difficult to confirm in the two-dimensional image from the camera provided on the catheter, through the three-dimensional CT image and carry out catheter treatment.

It is to be noted that, although it is described in the present specification that the two-dimensional image picked up by the image pickup element of the catheter is used, the present invention may be used not only for the two-dimensional image acquired directly from the image pickup element but also for other two-dimensional images. For example, the present invention may be used for individual two-dimensional images by an apparatus which involves a plurality of two-dimensional images as in the case of a 3D endoscope or may be used for a two-dimensional image produced from individual two-dimensional images.

Further, the method of detecting the position, the direction and the posture in the body of the catheter inserted in the body of the subject by the detection section is not limited to the method described hereinabove, but any method which is used generally by those skilled in the art can be used.

What is claimed is:

1. A catheter insertion supporting system, comprising:
an extraction section configured to extract a contour shape of at least one of organs of a subject represented on a two-dimensional image picked up by an image pickup element of a catheter inserted in the body of the subject;
a detection section configured to detect, in a three-dimensional computed tomography image of the subject, a cross section which includes a contour shape which at least partly coincides with the contour shape extracted by the extraction section; and
a display section configured to display a portion of the three-dimensional computed tomography image which includes an organ having the cross section detected by the detection section together with the two-dimensional image.

2. The catheter insertion supporting system according to claim 1, further comprising a correspondence relationship specification section configured to associate the organ having the cross section detected by the detection section in the three-dimensional computed tomography image and an organ having the contour shape in the two-dimensional image which at least partly coincides with the contour shape included in the cross section with each other to specify a correspondence relationship between each organ in the three-dimensional computed tomography image and each organ represented in the two-dimensional image,
the display section further displaying the correspondence relationship specified by the correspondence relationship specification section.

3. The catheter insertion supporting system according to claim 2, wherein the display section displays the correspondence relationship by color coding for the individual organs having a correspondence relationship to each other.

4. A catheter insertion supporting method, comprising:
an extraction step of extracting a contour shape of at least one of organs of a subject represented on a two-dimensional image picked up by an image pickup element of a catheter inserted in the body of the subject;
a detection step of detecting, in a three-dimensional computed tomography image of the subject, a cross section which includes a contour shape which at least partly coincides with the contour shape extracted at the extraction step; and
a display step of displaying a portion of the three-dimensional computed tomography image which includes an organ having the cross section detected at the detection step together with the two-dimensional image of the organs.

5. The catheter insertion supporting method according to claim 4, further comprising a correspondence relationship specification step of associating the organ having the cross section detected at the detection step in the three-dimensional computed tomography image and an organ having the contour shape in the two-dimensional image which at least partly coincides with the contour shape included in the cross section with each other to specify a correspondence relationship between each organ in the three-dimensional computed tomography image and each organ represented in the two-dimensional image,
the display step further displaying the correspondence relationship specified at the correspondence relationship specification step.

6. The catheter insertion supporting method according to claim 5, wherein the display step displays the correspondence relationship by color coding for the individual organs having a correspondence relationship to each other.

7. A non-transitory computer-readable storage medium stored with a program for a catheter insertion supporting system, the program causing the catheter insertion supporting system to execute a process comprising: an extraction step of extracting a contour shape of at least one of organs of a subject represented on a two-dimensional image picked up by an image pickup element of a catheter inserted in the body of the subject;
a detection step of detecting, in a three-dimensional computed tomography image of the subject, a cross section which includes a contour shape which at least partly coincides with the contour shape extracted at the extraction step; and
a display step of displaying a portion of the three-dimensional computed tomography image which includes an organ having the cross section detected at the detection step together with the two-dimensional image of the organs.

8. The non-transitory computer-readable storage medium according to claim 7, wherein the program further causes the catheter insertion supporting system to execute a correspondence relationship specification step of associating the organ having the cross section detected at the detection step in the three-dimensional computed tomography image and an organ having the contour shape in the two-dimensional image which at least partly coincides with the contour shape included in the cross section with each other to specify a correspondence relationship between each organ in the three-dimensional computed tomography image and each organ represented in the two-dimensional image,
the display step further displaying the correspondence relationship specified at the correspondence relationship specification step.

9. The non-transitory computer-readable storage medium according to claim 8, wherein the display step displays the correspondence relationship by color coding for the individual organs having a correspondence relationship to each other.

10. A catheter insertion supporting system, comprising:
a storage section configured to store a three-dimensional computed tomography image of a subject;
a detection section configured to detect a position and a direction of a catheter inserted in the body of the subject in the body;
a synthesis section configured to synthesize a three-dimensional image of an external form of the catheter with the three-dimensional computed tomography image stored in the storage section by combining the three-dimensional image into the three-dimensional computed tomography image such that a position and a direction of the external form of the catheter of the three-dimensional image in the three-dimensional computed tomography image coincide with a position and a direction in the body of the catheter detected by the detection section, respectively; and
a display section configured to display the three-dimensional computed tomography image synthesized by the synthesis section together with a two-dimensional image picked up by an image pickup element of the catheter.

11. The catheter insertion supporting system according to claim 10,
wherein the three-dimensional computed tomography image stored in the storage section has a marking applied thereto by a user, and
the display section displays the three-dimensional computed tomography image synthesized by the synthesis section and having the marking applied thereto.

12. The catheter insertion supporting system according to claim 10, wherein the display section displays the three-dimensional computed tomography image synthesized by the synthesis section so as to be rotatable by rotating an arbitrary coordinate axis of a coordinate system of the three-dimensional computed tomography image.

13. The catheter insertion supporting system according to claim 10,
wherein the detection section further detects a posture of the catheter inserted in the body of the subject in the body, and
the synthesis section synthesizes the three-dimensional image with the three-dimensional computed tomography image by combining the three-dimensional image into the three-dimensional computed tomography image such that the position, the direction and the posture of the external form of the catheter of the three-dimensional image in the three-dimensional computed tomography image coincide with the position, the direction and the posture of the catheter in the body detected by the detection section, respectively.

14. The catheter insertion supporting system according to claim 13, wherein the three-dimensional image to be combined into the three-dimensional computed tomography image by the synthesis section is selected from a plurality of three-dimensional images produced and stored in advance in the storage section and having different postures of the catheter or is produced based on the posture of the catheter detected by the detection section in the body.

15. A catheter insertion supporting method, comprising:
a storage step of storing a three-dimensional computed tomography image of a subject;
a detection step of detecting a position and a direction of a catheter inserted in the body of the subject in the body;
a synthesis step of synthesizing a three-dimensional image of an external form of the catheter with the three-dimensional computed tomography image stored at the storage step by combining the three-dimensional image into the three-dimensional computed tomography image such that a position and a direction of the external form of the catheter of the three-dimensional image in the three-dimensional computed tomography image coincide with a position and a direction in the body of the catheter detected at the detection step, respectively; and
a display step of displaying the three-dimensional computed tomography image synthesized at the synthesis step together with a two-dimensional image picked up by an image pickup element of the catheter.

16. The catheter insertion supporting method according to claim 15,
wherein the three-dimensional computed tomography image stored at the storage step has a marking applied thereto by a user, and
the display step displays the three-dimensional computed tomography image synthesized at the synthesis step and having the marking applied thereto.

17. The catheter insertion supporting method according to claim 15, wherein the display step displays the three-dimensional computed tomography image synthesized at the synthesis step so as to be rotatable by rotating an arbitrary coordinate axis of a coordinate system of the three-dimensional computed tomography image.

18. The catheter insertion supporting method according to claim 15,
wherein the detection step further detects a posture of the catheter inserted in the body of the subject in the body, and
the synthesis step synthesizes the three-dimensional image with the three-dimensional computed tomography image by combining the three-dimensional image into the three-dimensional computed tomography image such that the position, the direction and the posture of the external form of the catheter of the three-dimensional image in the three-dimensional computed tomography image coincide with the position, the direction and the posture of the catheter in the body detected at the detection step, respectively.

19. The catheter insertion supporting method according to claim 18, wherein the three-dimensional image to be combined into the three-dimensional computed tomography image at the synthesis step is selected from a plurality of three-dimensional images produced and stored in advance at the storage step and having different postures of the catheter or is produced based on the posture of the catheter detected at the detection step in the body.

20. A non-transitory computer-readable storage medium stored with a program for a catheter insertion supporting system, the program causing the catheter insertion supporting system to execute a process comprising:
a storage step of storing a three-dimensional computed tomography image of a subject;
a detection step of detecting a position and a direction of a catheter inserted in the body of the subject in the body;
a synthesis step of synthesizing a three-dimensional image of an external form of the catheter with the three-dimensional computed tomography image stored at the storage step by combining the three-dimensional image into the three-dimensional computed tomography image such that a position and a direction of the external form of the catheter of the three-dimensional image in the three-dimensional computed tomography image coincide with a position and a direction in the body of the catheter detected at the detection step, respectively; and
a display step of displaying the three-dimensional computed tomography image synthesized at the synthesis step together with a two-dimensional image picked up by an image pickup element of the catheter.

21. The non-transitory computer-readable storage medium according to claim 20,
wherein the three-dimensional computed tomography image stored at the storage step has a marking applied thereto by a user, and
the display step displays the three-dimensional computed tomography image synthesized at the synthesis step and having the marking applied thereto.

22. The non-transitory computer-readable storage medium according to claim 20, wherein the display step displays the three-dimensional computed tomography image synthesized at the synthesis step so as to be rotatable by rotating an arbitrary coordinate axis of a coordinate system of the three-dimensional computed tomography image.

23. The non-transitory computer-readable storage medium according to claim 20,
wherein the detection step further detects a posture of the catheter inserted in the body of the subject in the body, and
the synthesis step synthesizes the three-dimensional image with the three-dimensional computed tomography image by combining the three-dimensional image into the three-dimensional computed tomography image such that the position, the direction and the posture of the external form of the catheter of the three-dimensional image in the three-dimensional computed tomography image coincide with the position, the direction and the posture of the catheter in the body detected at the detection step, respectively.

24. The non-transitory computer-readable storage medium according to claim 23, wherein the three-dimensional image to be combined into the three-dimensional computed tomography image at the synthesis step is selected from a plurality of three-dimensional images produced and stored in advance at the storage step and having different postures of the catheter or is produced based on the posture of the catheter detected at the detection step in the body.

25. A calibration method, comprising:
an extraction step of extracting a contour shape of at least one of organs of a subject represented in a two-dimensional image picked up by an image pickup element of a catheter inserted in the body of the subject;
a detection step of detecting, in a three-dimensional computed tomography image of the subject, a cross section having a contour shape which at least partly coincides with the contour shape extracted at the extraction step;
a calculation step of calculating a relative positional relationship between the organ having the cross section detected in the three-dimensional computed tomography image at the detection step and the image pickup element of the catheter based on the two-dimensional image and specifications of a field of view of the image pickup element; and
a calibration step of specifying a position of the image pickup element of the catheter on a coordinate system of the three-dimensional computed tomography image based on the positional relationship calculated at the calculation step and the position of the organ in the three-dimensional computed tomography image thereby to adapt the coordinate system of the three-dimensional computed tomography image and the position of the image pickup element of the catheter to each other.

26. The calibration method according to claim 25, further comprising a display step of displaying a portion of the three-dimensional computed tomography image which includes the organ having the cross section detected at the detection step together with the two-dimensional image of the organ and displaying the position of the image pickup element of the catheter such that the position of the image pickup element of the catheter on the coordinate system of the three-dimensional computed tomography image specified at the calibration step can be specified in the three-dimensional computed tomography image.
